(19) United States
(12) Patent Application Publication
(10) Pub. No.: US 2008/0064905 A1
(43) Pub. Date: Mar. 13, 2008

(54) PROCESS FOR PREPARING 1,2-DIOLS FROM CARBONYL COMPOUNDS

(75) Inventors: Hans-Peter Krimmer, Kirchweidach (DE); Jurgen Sans, Trostberg (DE); Christoph Theis, Niederkassel (DE); Franz Thalhammer, Trostberg (DE)

Correspondence Address: OBLON, SPIVAK, MCCLELLAND MAIER & NEUSTADT, P.C., 1940 DUKE STREET, ALEXANDRIA, VA, 22314, US

(73) Assignee: DEGUSSA GmbH, Duesseldorf (DE)

(21) Appl. No.: 11/843,384

(22) Filed: Aug. 22, 2007

(30) Foreign Application Priority Data
Sep. 7, 2006 (DE) 102006041941.3-43

(51) Int. Cl. C07C 31/20

(52) U.S. Cl. 568/852

(57) ABSTRACT

1,2-diols can be obtained in good yields and in very high purity by a process of a)
reacting a carbonyl compound of the general formula (I) with hydrocyanic acid to give the corresponding cyanohydrin,

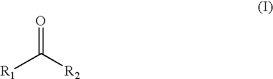

wherein R¹ and R² are each independently H, an optionally substituted straight-chain or branched C₁-C₁₈-alkyl radical, or an optionally substituted phenyl or C₅-C₆-cycloalkyl radical, b) subjecting the cyanohydrin obtained in process step a) to an acidic hydrolysis, and c) catalytically hydrogenating the 2-hydroxycarboxylic acid obtained from process step b) in the presence of a noble metal catalyst comprising ruthenium and rhenium.

### BACKGROUND OF THE INVENTION

1. Field of the Invention

The present invention relates to a three-stage process for preparing a 1,2-diol starting from a carbonyl compound.

2. Description of the Related Art

The reaction of carbonyl compounds such as aldehydes and ketones with hydrocyanic acid in the presence of basic catalysts is sufficiently well known according to the background art (cf., for example, the review “Formations of Cyanhydrins” in Science of Synthesis (2004) 19, 235-284).

For the subsequent hydrolysis of the nitrile group of the cyanohydrin, numerous processes have also already been described, and this hydrolysis can be performed in the acidic or alkaline pH range.

The acidic hydrolysis here forms 2-hydroxycarboxylic acids, while the alkaline hydrolysis also forms amino acid salts. For the acidic hydrolysis, according to the background art, various mineral acids or acidic ion exchangers are suitable. For example, the preparation of hydroxycarboxylic acid by acidic hydrolysis from the corresponding cyanohydrin is disclosed in JP H05-155 816 A. A further example of the acidic hydrolysis of cyanohydrins to 2-hydroxycarboxylic acids is described in the patent SU 1011630 A.

For the final catalytic hydrogenation of the 2-hydroxycarboxylic acids, according to the background art, various catalyst systems are available.

For instance, WO 01/16 063 recommends the hydrogenation of 2-hydroxycarboxylic acid with copper catalysts under very mild conditions and low pressures. WO 2005/077 870 provides the hydrogenation of 2-hydroxycarboxylic acids with the aid of doped noble metal catalysts. In addition, WO 2003/093 208 discloses the use of ruthenium catalysts for the reduction of carboxylic acids. Moreover, catalyst systems based on ruthenium and rhenium for the hydrogenation of 2-hydroxycarboxylic acids are disclosed in the international patent applications WO 99/38 824 and WO 99/38 613.

Finally, the patent DE 32 42 749 C1 describes a process for preparing 1,2-diols, wherein first a) the hydrogenation of a cyanohydrin at a hydrogen pressure of less than 10 bar in the presence of a palladium or platinum catalyst or in the presence of metallic nickel and of an organic or inorganic acid (for example hydrochloric acid) or of an acidic ion exchanger is continued until 1 mol of hydrogen has been taken up per mole of cyanohydrin used and then b) the hydrogenation is continued at a hydrogen pressure of 10 to 150 bar until the hydrogen uptake has ended. In this way, the corresponding diols can be prepared in good yields. However, a disadvantage in this process is the fact that the recovery and reactivation of the catalyst in the presence of the acidic ion exchanger is technically very complicated and costly. The regeneration of the ion exchanger in the presence of the ruthenium/rhenium catalyst is also likewise found to be technically difficult.

Finally, working with hydrochloric acid in the process according to DE 32 42 749 C1 entails considerable material problems, since the hydrogenation is performed in the presence of chloride ions and this reaction stage, owing to the associated corrosion problems, can therefore be effected only in autoclaves made from special materials.

### SUMMARY OF THE INVENTION

It was therefore an object of the present invention to develop a process for preparing 1,2-diols from carbonyl compounds which does not have the disadvantages of the background art mentioned, but rather provides the preparation of 1,2-diols in good yields and simultaneously—with regard to the performance and workup—can be performed in a technically simple manner.

This and other objects have been achieved by the present invention the first embodiment of which includes a process for preparing a 1,2-diol from a carbonyl compound, comprising:
a) reacting a carbonyl compound of the general formula (I) with hydrocyanic acid to give the corresponding cyanohydrin,

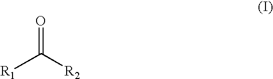

wherein

R¹ and R² are each independently H, an optionally substituted straight-chain or branched C₁-C₁₈-alkyl radical, or an optionally substituted phenyl or C₅-C₆-cycloalkyl radical,
b) subjecting the cyanohydrin obtained in process step a) to an acidic hydrolysis, and
c) catalytically hydrogenating the 2-hydroxycarboxylic acid obtained from process step b) in the presence of a noble metal catalyst comprising ruthenium and rhenium.

### DETAILED DESCRIPTION OF THE INVENTION

In accordance with the invention a process is provided for preparing 1,2-diols by a) firstly reacting a carbonyl compound of the general formula (I)

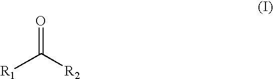

in which

R¹ and R² are each, independently, H, an optionally substituted straight-chain or branched C₁-C₁₈-alkyl radical, an optionally substituted phenyl or C₅-C₆-cycloalkyl radical, with hydrocyanic acid to give the corresponding cyanohydrin,

b) subjecting the cyanohydrin obtained from process step a) to an acidic hydrolysis, and

c) catalytically hydrogenating the 2-hydroxycarboxylic acid obtained from process step b) with the aid of a noble metal catalyst based on ruthenium and rhenium.

It has been found, surprisingly, that it is possible in this way to prepare 1,2-diols in good yields and in a technically simple manner (i.e. without special apparatus). Moreover, the 1,2-diols prepared in accordance with the invention have a very high purity, which was likewise not foreseeable.

The process according to the present invention comprises the three reaction stages a), b) and c). In the first reaction stage a), a carbonyl compound of the general formula (I)

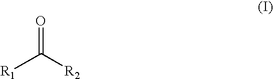

is reacted with hydrocyanic acid to give the corresponding cyanohydrin. In the general formula (I), R₁ and R₂ are each independently H, an optionally substituted straight-chain or branched C₁-C₁₈-alkyl radical or else an optionally substituted phenyl or C₅-C₆-cycloalkyl radical. In the case of alkyl or cycloalkyl radicals, the carbonyl compound may also have at least one substituent from the group of OH, NH₂ or OR³ where R³ is a C₁-C₈-alkyl radical. In the case of phenyl radicals, the carbonyl compound of the general formula (I) may also have at least one OH or NH₂ substituent.

In one embodiment, the carbonyl compounds are aldehydes and especially butyraldehyde.

The temperatures in reaction stage a) can be varied within wide limits, but it has been found to be particularly advantageous for reasons of economic viability to perform this reaction at temperatures of 0 to 100° C. and especially 0 to 30° C. The reaction temperature includes all values and subvalues therebetween, especially including 10, 20, 30, 40, 50, 60, 70, 80 and 90° C. It is particularly advantageous to perform reaction stage a) without solvent or in the presence of a solvent selected from the group of water, alcohols, ethers or mixtures thereof, preference being given to using C₁-C₄ alcohols among the alcohols, and diethyl ether and THF among the ethers.

Moreover, process stage a) can be performed in the presence of a basic catalyst, and organic amines, for example triethylamine, can preferably be employed. The basic catalyst is preferably used in an amount of 0.1 to 10 mol % based on the carbonyl compound. The amount of basic catalyst includes all values and subvalues therebetween, especially including 0.5, 1, 2, 3, 4, 5, 6, 7, 8, 9 mol %.

In one embodiment, a 0.1 to 10% molar excess of hydrocyanic acid is employed in reaction stage a), based on the carbonyl compound used. The molar excess of hydrocyanic acid includes all values and subvalues therebetween, especially including 0.5, 1, 2, 3, 4, 5, 6, 7, 8, 9 mol %.

On completion of reaction stage a), it is the cyanohydrin formed can be stabilized by adjusting the pH of the reaction mixture to 1.0 to 6.0 with the aid of acids, for example hydrochloric acid. The pH includes all values and subvalues therebetween, especially including 1.5, 2, 2.5, 3, 3.5, 4, 4.5, 5 and 5.5.

In the subsequent second reaction stage b), the cyanohydrin obtained from process step a) is subjected to an acidic hydrolysis. The acidic hydrolysis is preferably performed with the aid of a mineral acid, especially in the form of an aqueous solution, or in the presence of an acidic ion exchanger.

A useful mineral acid here has been found in particular to be 20 to 37% hydrochloric acid or 50 to 80% sulphuric acid. The ratio of mineral acid to cyanohydrin is relatively uncritical, but it has been found to be particularly advantageous to use the mineral acid in an acid equivalent ratio to the cyanohydrin of 1.0 to 10.0:1 and more preferably of 1.2 to 2.0:1.

The hydrolysis step b) is performed preferably at elevated temperatures of 30 to 130° C. and in particular of 60 to 110° C. The temperature includes all values and subvalues therebetween, especially including 40, 50, 60, 70, 80, 90, 100, 110 and 120° C.

It is immediately possible in the context of the present invention to perform the hydrolysis under elevated pressures, preference being given to boiling conditions under standard pressure.

On completion of the acidic hydrolysis, in one embodiment, the pH of the reaction mixture is adjusted in aqueous sodium hydroxide solution to a pH of 0 to 4, the solvent is removed and the 2-hydroxycarboxylic acid is obtained by crystallization or extraction with an organic solvent. The pH includes all values and subvalues therebetween, especially including 0.5, 1, 1.5, 2, 2.5, 3, 3.5. In the case of extraction, the 2-hydroxycarboxylic acid is preferably subsequently purified by distillation under reduced pressure at 0.1 to 100 mbar. The pressure includes all values and subvalues therebetween, especially including 0.5, 1, 5, 10, 20, 30, 40, 50, 60, 70, 80, 90 mbar.

In the third reaction stage c), the 2-hydroxycarboxylic acid obtained from process step b) is catalytically hydrogenated with the aid of a noble metal catalyst based on ruthenium and rhenium. In this case, the noble metal catalyst in stage c) has a preferred noble metal content of 1 to 10% by weight, the support material used preferably being activated carbon. The noble metal content includes all values and subvalues therebetween, especially including 2, 3, 4, 5, 6, 7, 8, 9% by weight.

Moreover, the ruthenium/rhenium catalyst used in the hydrogenation step c) has a preferred content of ruthenium and rhenium, in each case, of 1 to 10% by weight. The content of ruthenium and rhenium, in each case, includes all values and subvalues therebetween, especially including 2, 3, 4, 5, 6, 7, 8, 9% by weight.

The reaction conditions in the hydrogenation step c) can likewise be varied within wide limits. Particularly advantageous conditions have been found to be temperatures of 100 to 300° C. and more preferably of 180 to 200° C., and hydrogen pressures of 50 to 300 bar and more preferably of 200 to 250 bar. The temperature includes all values and subvalues therebetween, especially including 150, 200, 250° C. The pressure includes all values and subvalues therebetween, especially including 100, 150, 200, 250 bar.

In one embodiment, the hydrogenation stage c) can also be undertaken in two steps. The amount of the catalyst used is generally 0.1 to 7.5% by weight and especially 2 to 5% by weight, based in each case on the amount of 2-hydroxycarboxylic acid used. The amount of the catalyst includes all values and subvalues therebetween, especially including 0.5, 1, 1.5, 2, 2.5, 3, 3.5, 4, 4.5, 5, 5.5, 6, 6.5 and 7% by weight

Depending on the reaction conditions, the hydrogenation in reaction stage c) has ended after at most 20 hours, preferably after 1 to 12 hours and more preferably after 7 to 10 hours.

On completion of reaction stage c), the ruthenium/rhenium catalyst is removed from the crude product by filtration and can, in one embodiment, be recycled.

The resulting reaction product comprising the corresponding 1,2-diol can, for example, be isolated or purified by distillation. In the process of the present invention, the 1,2-diol is obtained in good yields of >60% and very high purities of >98%. The yield includes all values and subvalues therebetween, especially including 65, 70, 75, 80, 85, 90, 95, 97, 98, 99 and 99.5%. The purity includes all values and subvalues therebetween, especially including 98.5, 99, 99.5%.

In the process according to the invention, 1,2-pentanediol is preferably obtained.

Having generally described this invention, a further understanding can be obtained by reference to certain specific examples which are provided herein for purposes of illustration only, and are not intended to be limiting unless otherwise specified.

### EXAMPLES

### Example 1

A glass flask was initially charged with 72 g (1.0 mol) of butyraldehyde at room temperature and mixed with 0.5 g of triethylamine as a catalyst. 27.5 g (1.02 mol) of hydrocyanic acid were then metered in at 15-20° C. under temperature control and stirred at room temperature for about 1 hour. Hydrochloric acid was used to establish a pH of 2-4 in order to stabilize the cyanohydrin formed.

A second flask was initially charged with 243 g (2.0 mol) of 30% hydrochloric acid at 50° C., and the cyanohydrin from flask 1 was metered in within 30 minutes. During the addition, the temperature was increased up to reflux (at 106° C.). Thereafter, the mixture was stirred under reflux for another 1 hour. The acid excess was then neutralized with 40% aqueous sodium hydroxide solution up to pH 2.

The resulting reaction mixture was admixed with 200 ml of methyl tert-butyl ether and the aqueous phase was removed. In the organic phase, 2-hydroxypentanoic acid was present in a yield of >90% which, after the solvent had been distilled off, was distilled at 140-150° C. under a reduced pressure of 1 mbar. The yield was 73.8 g (71% of theory).

29.5 g (0.25 mol) of the 2-hydroxypentanoic acid thus obtained were dissolved in 470.5 g of water and this mixture, after adding 5.0 of water-moist catalyst on activated carbon (coating: 3% Ru and 2% Re), was hydrogenated exhaustively in a 11 autoclave at a hydrogen pressure of 250 bar and at a reaction temperature of 190° C. within 8 hours.

After the end of the reaction and cooling, the catalyst was filtered off and the filtrate was freed of water by distillation under reduced pressure using a rotary evaporator with a column attachment.

The resulting residue was then, using a short-path evaporator with a short column, subjected to a fractional high-vacuum distillation.

This afforded 16.8 g of 1,2-pentanediol with a GC purity (after silylation) of 98.7%, which corresponds to a yield of 64.6% of theory based on 2-hydroxypentanoic acid used.

### Example 2

A glass flask was initially charged with 360 g (5.0 mol) of butyraldehyde and 300 ml of water at room temperature, and mixed with 2.5 g of triethylamine as a catalyst. 137 g (5.1 mol) of hydrocyanic acid were then metered in at 15-20° C. under temperature control and the mixture was stirred at room temperature for about 1 hour. 80% sulphuric acid was used to establish a pH of 2-4 in order to stabilize the cyanohydrin formed.

A second flask was initially charged with 980 g (8.0 mol) of 80% sulphuric acid at 90° C., and the cyanohydrin from flask 1 was metered in within 60 minutes. During the addition, the temperature was increased to 105-110° C. Thereafter, the mixture was stirred at this temperature for another 3 hours. After cooling, the acid excess was neutralized with 40% aqueous sodium hydroxide solution up to pH 2.

The resulting reaction mixture was admixed with 1000 ml of methyl tert-butyl ether and the aqueous phase was removed. In the organic phase, 2-hydroxypentanoic acid was present together with approx. 10% unhydrolyzed butyraldehyde cyanohydrin.

After the solvent had been distilled off, the product was distilled together with the cyanohydrin at 140-150° C. under a reduced pressure of 1 mbar. The yield was 338.0 g (51.5% of theory based on a purity of 90%).

A 1 l autoclave was charged with a suspension consisting of 400 g of water and 5.0 g of water-moist catalyst (coating: 8% Ru and 1% Re) and prehydrogenated at 190° C. and 250 bar of hydrogen for the period of 2 hours.

Subsequently, after cooling and decompression, 156.0 g of 2-hydroxypentanoic acid—which still contains a proportion of approx. 10% of butyraldehyde cyanohydrin—were added to the catalyst suspension and the reaction mixture was subsequently hydrogenated at a reaction temperature of 190° C. and a hydrogen pressure of 250 bar over the course of 14 hours until the hydrogen uptake had ended.

After catalyst removal, the conversion of the hydrogenation was determined to be 96% by acidimetric titration; the further distillative workup of the filtrate was effected analogously to Example 1.

This afforded 89.2 g of 1,2-pentanediol with a purity of 98.1%; this corresponds—based on 2-hydroxypentanoic acid used—to a yield of 72.1% of theory.

### Example 3

A glass flask was initially charged with 72 g (1.0 mol) of butyraldehyde at room temperature and mixed with 0.5 g of triethylamine as a catalyst. 27.5 g (1.02 mol) of hydrocyanic acid were then metered in at 15-20° C. under temperature control and stirred at room temperature for about 1 hour. Hydrochloric acid was used to establish a pH of 2-4 in order to stabilize the cyanohydrin formed.

A second flask was initially charged with 243 g (2.0 mol) of 30% hydrochloric acid at 50° C. and the cyanohydrin from flask 1 was metered in within 30 minutes. During the addition, the temperature was increased up to reflux (at 106° C.). Thereafter, the mixture was stirred under reflux for another 1 hour. The acid excess was then neutralized with 40% aqueous sodium hydroxide solution up to pH 2.

The resulting reaction mixture was admixed with 200 ml of methyl tert-butyl ether and the aqueous phase was removed. The organic phase was extracted by shaking with 200 ml of water and removed from the water phase. It contained 2-hydroxypentanoic acid in a yield of >90%. The organic solvent was removed by distillation and the residue was subjected to hydrogenation (stage c).

To this end, a 1 l autoclave was charged with a suspension consisting of 400 g of water and 5.0 g of water-moist catalyst (coating: 8% Ru and 1% Re) and prehydrogenated at 190° C. and 250 bar of hydrogen for a period of 2 hours.

Subsequently, after cooling and decompression, the crude 2-hydroxypentanoic acid was added to the catalyst suspension and the reaction mixture was subsequently hydrogenated at a reaction temperature of 190° C. and a hydrogen pressure of 250 bar over the course of 14 hours until the hydrogen uptake had ended.

After catalyst removal, the conversion of the hydrogenation was determined to be 96% in the filtrate by acidimetric titration; the further distillative workup of the filtrate was effected analogously to Example 1.

This afforded 68.6 g of 1,2-pentanediol with a purity of 98.1%; this corresponds—based on butyraldehyde used—to a yield of 66.0% of theory.

German patent application 10 2006 041 941.3-43 filed Sep. 7, 2006, is incorporated herein by reference.

Numerous modifications and variations on the present invention are possible in light of the above teachings. It is therefore to be understood that within the scope of the appended claims, the invention may be practiced otherwise than as specifically described herein.

1. A process for preparing a 1,2-diol from a carbonyl compound, comprising:
a) reacting a carbonyl compound of the general formula (I) with hydrocyanic acid to give the corresponding cyanohydrin, 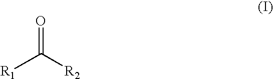
wherein
R¹ and R² are each independently H, an optionally substituted straight-chain or branched C₁-C₁₈-alkyl radical, or an optionally substituted phenyl or C₅-C₆-cycloalkyl radical,
b) subjecting the cyanohydrin obtained in process step a) to an acidic hydrolysis, and
c) catalytically hydrogenating the 2-hydroxycarboxylic acid obtained from process step b) in the presence of a noble metal catalyst comprising ruthenium and rhenium.

2. The process according to claim 1, wherein R¹ and R², in the case of alkyl or cycloalkyl radicals, have at least one substituent selected from the group consisting of OH, NH₂ and OR³ (wherein R³═C₁-C₈-alkyl), and
wherein R¹ and R², in the case of phenyl radicals, have at least one substituent selected from the group consisting of OH and NH₂.

3. The process according to claim 1, wherein reaction stage a) is performed at temperatures of 0 to 100° C.

4. The process according to claim 1, wherein stage a) is performed in the presence of a solvent selected from the group consisting of water, alcohols, ethers and mixtures thereof.

5. The process according to claim 1, wherein the hydrocyanic acid is used in stage a) in a 5 to 10% molar excess based on the carbonyl compound.

6. The process according to claim 1, wherein process step a) is performed in the presence of a basic catalyst.

7. The process according to claim 1, wherein from 0.1 to 10 mol % of a basic catalyst, based on the carbonyl compound, are used in step a).

8. The process according to claim 1, wherein on completion of reaction stage a), the cyanohydrin is stabilized by adjusting the pH of the reaction mixture to 1.0 to 6.0.

9. The process according to claim 1, wherein the acidic hydrolysis in process step b) is performed in the presence of a mineral acid and/or an acidic ion exchanger.

10. The process according to claim 9, wherein the mineral acid is used in an acid equivalent ratio relative to the cyanohydrin of from 1.0 to 10.0:1.

11. The process according to claim 1, wherein the hydrolysis step b) is performed at a temperature of from 30 to 130° C. optionally under elevated pressures, preference being given to boiling conditions under standard pressure.

12. The process according to claim 1, wherein before the hydrogenation step c), the 2-hydroxycarboxylic acid is purified by crystallization or extraction with an organic solvent, optionally after increasing the pH with aqueous sodium hydroxide solution to a pH of 0 to 4, and removing the solvent.

13. The process according to claim 1, wherein the 2-hydroxycarboxylic acid is purified by distillation under reduced pressure at 0.1 to 100 mbar.

14. The process according to claim 1, wherein the ruthenium/rhenium catalyst in the hydrogenation step c) has a noble metal content of 1 to 10% by weight.

15. The process according to claim 1, wherein the noble metal catalyst in stage c) has a content of ruthenium of 1 to 10% by weight and a content of rhenium of 1 to 10% by weight.

16. The process according to claim 1, wherein a support material of the ruthenium/rhenium catalyst comprises activated carbon.

17. The process according to claim 1, wherein the hydrogenation is performed at a temperature of from 100 to 300° C.

18. The process according to claim 1, wherein the hydrogenation step c) is performed at hydrogen pressures of 50 to 300 bar.

19. The process according to claim 1, wherein the hydrogenation is performed over a period of not more than 20 hours.

20. The process according to claim 1, wherein the amount of catalyst used is 0.1 to 7.5% by weight, based on the amount of 2-hydroxycarboxylic acid.

21. The process according to claim 1, wherein the hydrogenation catalyst is recycled.

22. The process according to claim 1, wherein said 1,2-diol is isolated and/or purified by distillation.

23. The process according to claim 1, wherein said 1,2-diol is 1,2-pentanediol.

24. The process according to claim 1, wherein process step a) is performed in the presence of an organic amine.

25. The process according to claim 1, wherein the acidic hydrolysis in process step b) is performed in the presence of 20 to 37% hydrochloric acid or 50 to 80% sulphuric acid.

26. The process according to claim 1, wherein the hydrolysis step b) is performed under boiling conditions of the solvent and under standard pressure.
